(19) United States
(12) Patent Application Publication
(10) Pub. No.: US 2007/0252106 A1
(43) Pub. Date: Nov. 1, 2007

(54) Intermediate Product Comprised of a Mixture of Organic Carbonates and Carbamates and a Method for Its Manufacture

(76) Inventors: Henning Buchold, Hanau (DE); Jurgen Eberhardt, Rodgau (DE); Ulrich Wagner, Biendorf (DE); Hans-Jorg Wolk, Rosenheim (DE)

Correspondence Address: K.F. ROSS P.C., 5683 RIVERDALE AVENUE, SUITE 203 BOX 900, BRONX, NY, 10471-0900, US

(21) Appl. No.: 10/571,997

(22) PCT Filed: Jul. 16, 2004
(86) PCT No.: PCT/EP04/07912

§ 371 Date: Mar. 9, 2007

(30) Foreign Application Priority Data
Sep. 11, 2003 (DE) 103 41 952.7

(51) Int. Cl. C07C 69/96; C07C 271/12; C07D 323/00; C08G 65/333; C07C 68/00

(52) U.S. Cl. 252/182.3; 252/182.31

(57) ABSTRACT

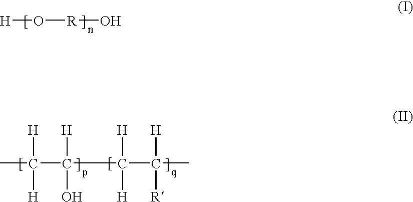

The invention relates to an intermediate product based on the organic carbonates and carbamates mixture and method for the production thereof. The inventive method consists in reacting a carbamide, a substituted carbamide, a carbamic acid salt or ester or one of the N-substituted derivatives thereof in a glycol polyalkylene, polyol polyester or a polyol polyether of general formula (I), wherein R is a linear or branched alkylene group having from 2 to 12 carbon atoms and n is a number ranging from 1 to 20, or in a totally or partly hydrolysed polyvinyl alcohol of general formula (II), wherein R′ is an alkyl, aryl or acyl group having from 1 to 12 carbon atoms p and q are numbers ranging from 1 to 20, or dissolving in the mixture of said compounds with an alkaline catalyst promoting the ammonia separation. The thus free ammonia or an amine produced by the reaction mixture is removed by a stripping gas.

The object of the invention is an intermediate product comprising a mixture of organic carbonates and carbamates, which is a valuable starting material for the manufacture of organic carbonates, as well as a method for the manufacture of this intermediate product.

Dimethyl carbonate and diphenyl carbonate are intermediates in the chemical industry, which are employed in a multiplicity of application areas. Thus dimethyl carbonate in a starting material for aromatic polycarbonates. Dimethyl carbonate is transesterified with phenol to diphenyl carbonate and converted in a melt polymerization with bisphenol to the aromatic polycarbonate (Daniele Delledonne; Franco Rivetti; Ugo Romano: “Developments in the Production and Application of Dimethyl Carbonate” Applied Catalysis A: General 221 (2001) 241-251). Dimethyl carbonate be employed for improvement of the octane number of gasoline and substituted for environmentally troublesome additives like MTBE (Michael A. Pacheco; Christopher L. Marshall: “Review of Dimethyl Carbonate (DMC) Manufacture and its Characteristics as a Fuel Additive” Energy and Fuels 11 (1997) 2-29. In this connection above all the easy biodegradability, the non-toxicity and the good applicability as a gasoline additive is to be mentioned. Dimethyl carbonate has a range of applications in chemical synthesis. At temperatures at or under the boiling point of 90° C. dimethyl carbonate can be used as a methoxylating agent. At higher temperatures around 160° C. dimethyl carbonate can be employed as a methylating agent (Pietro Tundi; Maurizio Selva: “The Chemistry of Dimethyl Carbonate” Acc. Chem. Res. 35 (2002) 706-716).

Up to about 1980 the method used for the manufacture of dimethyl carbonate was the alcoholysis of phosgene with methanol (U.S. Pat. No. 2,379,740, Pittsburgh Plate Glass Company 1941) or (Kirk-Othmer, Encyclopedia of Chemical Technology, 3^{rd} Edition, Volume 4, 758). The toxicity of phosgene and formation of corrosive hydrogen chloride of course militate against an environmentally conscious commercial use on a large scale.

To date the mainly utilized process is the conversion of methanol with carbon monoxide and oxygen on a copper chloride contact, described in U.S. Pat. No. 5,210,269 from Enichem (1993). This oxidative carbonylation involves running over copper methoxy chloride and a subsequent reaction with carbon monoxide to dimethyl carbonate. The main problem of this process is the deactivation of the catalysts by water. The- deactivated catalyst must be expensively regenerated or the water content in the reactor be held low.

A variant of the oxidative carbonylation is a two-stage reaction over methyl nitrite. In a pre-reactor methyl nitrite is synthesized from methanol, nitric oxide and oxygen, wherein water is formed as a byproduct. After removal of the water gaseous methyl nitrite is converted with CO to dimethyl carbonate in a solid bed reactor on a palladium chloride catalyst; The NO formed is fed into this circuit. This method has the disadvantage that the operation with corrosive nitric oxide is dangerous.

Another possibility for the manufacture of dimethyl carbonate is the transesterification of a cyclic carbonate with methanol. Methods with ethylene or propylene carbonate as starting material are known (U.S. Pat. No. 4,734,518 Texaco 1988; U.S. Pat. No. 4,691,041 Texaco. 1987). Starting from the cyclic carbonate the dimethyl carbonate can be synthesized and simultaneously in each case one mole of the corresponding diol are synthesized. The alkylene carbonates can be simply prepared. The disadvantage of this method is the co-production of diols with the manufacture of dimethyl carbonate.

The direct alcoholysis of urea with methanol is another possibility from the manufacture of dimethyl carbonate. The synthesis goes in two steps via the carbamic acid methyl ester to the dimethyl carbonate. The reaction rate strongly inhibited through the ammonia that is formed. For the improved synthesis therefore chemical and physical methods were proposed to remove the ammonia which is formed.

Also a precipitation of the ammonia formed by means of BF₃ was successfully performed (U.S. Pat. No. 2,834,799, 1958), but is uneconomical in view of the higher salt loads arising.

The removal of ammonia (U.S. Pat. No. 4,436,668; BASF 1984) by addition of inert gas in a second stage furnishes up to now only unsatisfactory conversions and selectivities. For improvement of the process, a second stage was employed with a reacting catalyst diakyl isocyanatealkoxy tin (U.S. Pat. No. 5,565,603; Exxon 1996; U.S. Pat. No. 5,561,094; Exxon 1996), which is prepared in situ through methanol. As a disadvantage the preparation and processing of the reacting catalyst is to be mentioned.

An alternative to the direct synthesis is the operation of a cyclic carbonate (U.S. Pat. No. 5,489,702 Mitsubishi Gas Chemical 1996; U.S. Pat. No. 5,349,077; Mitsubishi Gas Chemical 1994). Here in a first step a diol is reacted with urea and a cyclic alkylene carbonate with 5 or 6 ring atoms is synthesized. In the second process step the alkylene carbonate is transesterified with methanol. The diol can subsequently be fed into the circuit.

The intermediate products prepared in the alcoholysis must subsequently be reacted with methanol, in order to obtain dimethyl carbonate as product. The transesterification is a catalyzed reaction. As heterogeneous catalysts basic alkali and alkaline earth metals or oxides are employed. Examples of alkaline or alkaline earth metals in zeolites are given in U.S. Pat. No. 6,365,787 from Exxon. Examples of metal oxides are named in U.S. Pat. No. 6,207,850 Mobil Oil. Methods for the transesterification of ethylene and propylene carbonates with alcohols in counter current solid bed tube reactors with homogeneous or heterogeneous catalysts (U.S. Pat. No. 5,231,212; Bayer 1993; U.S. Pat. No. 5,359,188; Bayer 1994) as well as a method patent for the synthesis by means of epoxides with subsequent transesterification on bifunctional catalysts (U.S. Pat. No. 5,218,135; Bayer 1993) are likewise known already. The transesterification of cyclic carbonates with alcohols in a reactive distillation is described (U.S. Pat. No. 6,346,638; Asahi Kasai Kabushiki Kaisha 2002). A reactive extraction with hydrocarbons or gasoline as phases for the absorption of dimethyl carbonate and a polar phase of alkylene carbonate for absorption of the alcohols is known from U.S. Pat. No. 5,489,703.

Only a few of these are in principle possible synthesis routes for a prospective engineering and economical realization. For the required large quantities of dimethyl carbonate only those methods come into consideration which also have the necessary inexpensive raw materials available in sufficient quantities. In recent years therefore the manufacture of organic carbonates preferably dimethyl carbonate, on the basis of urea and methanol, has been strenuously worked on to implement on an engineering scale. Despite numerous developments the methods described up to now partly possess significant disadvantages, so that an elegant engineering route for the production of organic carbonates, such as DMC is still lacking.

As disadvantageous the methods described up to now show:
The reaction of urea with methanol proceeds via the intermediate stage of carbamates.
During the reaction ammonia is split off, which must be removed.
Because of insufficient ammonia the reaction proceeds with only small degrees of conversion.
Ammonia can in principle be removed from the reaction mixture via different methods, however in the methods known from the state of the art, hereby a solid material to be disposed of forms or a large part of the methanol employed is also removed.
Large amounts of methanol must be utilized in the circuit.
A developed method for DMC can not without further ado be extended from the synthesis of other carbonates. A method which overcomes these disadvantages is described in the simultaneously filed patent application (internal reference L 1 P 21/20030014). In this method a polymeric intermediate product is employed, which has such a high boiling point, that it is also not removed in the necessary driving off of ammonia by stripping with gas or steam or by application of a vacuum. This intermediate product applicable for the manufacture of organic, aliphatic and aromatic dicarbonates is comprised of different organic carbonates and carbamates of polymeric alcohols and has the special advantage, that it can be a mixture whose properties can be adjusted by choice of the components and their optimal proportions for the requirements of the method. This intermediate product and its manufacture are the object of the present application.

The intermediate product is a mixture of organic carbonates and carbamates, which through reaction of urea, a substituted urea, a salt or ester of carbamic acid or one of its N-substituted derivatives (alkyl, aryl groups like methyl, ethyl, phenyl, benzyl)
with polymeric multifunctional alcohols like polyalkyleneglycols, polyester polyols or polyether polyols of the general formula I
   H—[—O—R—]ₙ—OH (formula I)
in which R stands for a straight chain or branched chain alkylene group having 2 to 12 carbon atoms and n is a number between 2 and 20,
or completely or partially hydrolyzed polyvinylalcohols of the general formula II 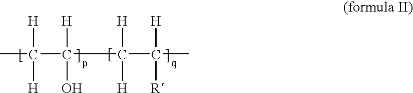
in which R′ stands for an alkyl, aryl or acyl group having 1-12 carbon atoms, p and q are numbers between 1 and 20
or with mixtures of these compounds, without or in presence of a favorable catalyst for splitting off ammonia and removal of ammonia or amines thereby liberated is obtainable at a reaction temperature of at least 100° C., preferably at about 200° C. and in a reaction time of about five hours.

The method in accordance with the invention for manufacture of a mixture of organic carbonates and carbamates, comprises therein that urea, a substituted urea, a salt or ester of carbamic acid or one of its N-substituted derivatives (alkyl, aryl groups like methyl, ethyl, phenyl, benzyl)
in a first stage with polymeric multifunctional alcohols like polyalkyleneglycols, polyester polyols or polyether polyols of the general formula I
   H—[—O—R—]ₙ—OH (formula I)
in which R stands for a straight chain or branched chain alkylene group having 2 to 12 carbon atoms and n is a number between-2 and 20,
or completely or partially hydrolyzed polyvinylalcohols of the general formula II 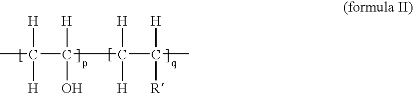
In which R′ stands for an alkyl, aryl or acyl group having 1-12 carbon atoms, p and q are numbers between 1 and 20,
or dissolved in mixtures of these compounds, without or in the presence of catalyst favoring splitting off of ammonia reacts to a carbonate and carbamate containing mixture, which thereby removes liberated ammonia or the amine is removed from the reaction mixture by means of a stripping gas and/or steam and/or vacuum and
In a second stage (transesterification) the carbonates and carbamates of the polymeric alcohol containing mixture is reacted with an alcohol or a phenol with formation of their carbonates and back formation of the polymeric polyalcohols of formulas I or II.

Up to now for the manufacture of the intermediate carbamate product according to the state of the art monomeric glycol and monomeric diols are utilized with urea (Michael A. Pacheco; Christopher L. Marshall: “Review of Dimethyl Carbonate (DMC) Manufacture and its Characteristics as a Fuel Additive” Energy & Fuels 11 (1997) 2-29). This is carried out in the first stage in order to produce therefrom the carbonates of these alcohols.

Surprisingly it has now been shown that the utilization of polymeric alcohols has a series of essential advantages in contrast to the state of the art.

Surprisingly it has been shown that the utilization of polymeric alcohols has a series of essential advantages in contrast to the state of the art.
Polymeric alcohols and the carbonates and carbamates formed from them possess an essentially higher boiling point than the monoalcohols, diols and the carbonates and carbamates formed from them described up until now in the state of the art. This results in that in the removal of ammonia produced by the reaction by stripping or vacuum an almost complete conversion and at the same time minimal losses of these higher boiling alcohols and carbonates or carbamates is achieved. This is not possible by use of the state of the art known methods, since upon stripping also large proportions of the alcohols used there and the glycol carbonates or diol carbonates are driven out of the reaction mixture.
Due to their water-like polar structure polymeric alcohols possess a higher solubility for urea, substituted ureas, salts and esters of carbamic acid and its N-substituted derivatives as the long chain monoalcohols or diols used up to now, so that the conversions can be carried out in homogeneous solution. Through changing the chain length n and the size of the subgroups R the solubility and at the same time the boiling point of the mixture desired can be set. Additionally polymeric alcohols are still liquid at temperatures at which comparably long monoalcohols or diols are already solid.
Furthermore these auxiliary materials have an adjustable viscosity are less corrosive and are consequently specially suited for a circuit way of operation. Further they are not toxic and consequently environmentally neutral.
Polymeric alcohols posses a distinctly higher chemical, thermal and mechanical stability than the materials used up to now, which for recovery (circuit operation) of these alcohols after the back formation in the second stage is a large advantage, since the losses of polymeric alcohols due to decomposition or thermal cracking processes are minimal.
Normally one would not consider employing polymeric alcohols for the purpose of intermediate formation because polymeric alcohols are multicomponent mixtures, which are more difficult to handle from an engineering standpoint than pure auxiliary materials. The multicomponent mixture produced when polymeric alcohols are employed caused as a rule by the further processing difficulties. However directly through the utilization of polymeric alcohols an engineering advantage is produced here. Because it has been surprisingly shown that it is not at all necessary to process the resulting multicomponent mixture, but that one can proceed directly to the second stage (transesterification) without disadvantages arising hereby. Because in the transesterification with lower alcohols or phenols, all initially present polymeric alcohols form back completely exactly as the usually used monoalcohols or diols. These can then be fed again to the first stage (circuit way of operating).

The advantages of the intermediate products manufactured according to the method in accordance with the invention are:
The intermediate products obtained have high boiling points whereby a wider, technically usable range for the free adjustment of pressure and temperature are available for the manufacture of different organic carbonates or carbamates;
Thereby the setting of a high stripping gas stream is possible for ammonia or amine removal.
Conversion of urea, substituted ureas, slats and esters of carbamic acid and their N-substituted derivatives with polymeric alcohols to high-boiling carbonates and carbamates in a single stage.
High conversions and yields by means of simultaneous removal (striping with gas and/or vapor of application of vacuum) of the ammonia produced in the reaction with minimal losses of alcohols, carbonates and carbamates.
The reaction does not necessitate a catalyst. However through the use of basic catalysts a further increase of the reaction rate can be achieved.

The effectiveness of the new process proposed here for the manufacture of organic carbonates or carbamates should be elucidated based upon a few examples.

The method in accordance with the invention is carried out in an advantageous way at temperatures between 107 and 270° C. Thereby under normal pressure or reduced pressure and dosage one of the suitable gases or vapor for driving out the ammonia formed in the presence of catalysts is operated. For this alkaline reacting salts, oxides, hydroxides, alcoholates of the first and second main group or of the 1 to 8 subgroup of the periodic system, basic zeolites or polymeric ion exchangers are suitable as catalysts. For example magnesium or zinc catalysts which can be employed as the oxide or also as well as the acetate can be catalytically active. An important influencing value is the removal of ammonia through stripping with gas, steam or vacuum.

In a second stage the mixture manufactured in accordance with the invention can be reacted further, e.g. can be converted with an alcohol or with a phenol in the presence of a basic catalyst, to manufacture an organic carbonate.

The invention is explained in detail by means of the following tests.

### Test Construction and Implementation

All tests for the transesterification of the urea dissolved in a polymeric alcohol were carried out in a 150 ml double-mantle glass reactor with a heating mantle, gassing device and reflux condenser. A drop separator before the entry into the reflux condenser prevented the discharge of carried over liquid. As stripping gas nitrogen was employed. Vacuum could be employed by means of an attached membrane pump. Samples were taken discontinuously.

### Investigations with Polyethyleneglycol

Polyethyleneglycol is a suitable reactant, since it has a range of interesting properties. The transesterification of the bivalent alcohol with urea can in principle produce two products. These two long-chain carbonates are:

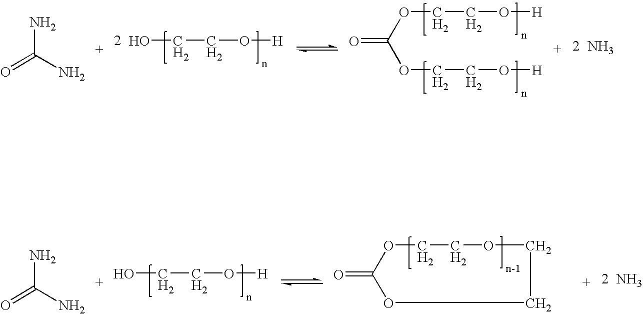

Both carbonates are suited for the transesterification with methanol in the second stage to the desired product. The investigations showed that the reaction to the cyclic carbonate is more likely, since the reaction takes place in a ratio of 1 mole urea to 1 mole polyethyleneglycol. In both cases the carbamate is to be observed as intermediate product:

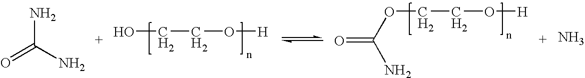

### Use of Different Catalysts

The catalysts named in the patent literature comprise a series of metal oxides. In the tests carried out in accordance with the invention oxide and acetate powders came to be employed. They were employed in mass ratios between 5 and 25 wt %. Titanium dioxide, zinc oxide, magnesium oxide-and magnesium acetate were investigated as possible catalysts.

In that connection only small differences in the course of the reaction course were displayed for these different catalysts. The reaction rate was also very low at 150° C. and even after 16 hours no end of the reaction was foreseeable. The acceleration of the reaction was almost equally large for magnesium acetate, magnesium oxide and zinc oxide. These compounds displayed a clearly better catalytic activity than titanium compounds.

An increase of the amount of catalyst was investigated but did not bring about the hoped for difference in the reaction rate. At 150° C. as good as no difference was discerned between the tests with 6 or 20 g magnesium acetate. Also at higher temperatures of 200° C., after an initially more rapid development of product no marked difference in the product quantities was to be discerned.

### Variation of the Temperature

Previous tests of reactions of urea with polyethyleneglycol have shown that below about 140° C. as good as no reaction is to be observed. Therefore 150° C. was chosen as minimal test temperature. In the tests with titanium dioxide a rather moderate nitrogen stream volume was used to drive off the ammonia. A clear influence of the reaction temperature in the course of the time dependant polyethyleneglycol concentration is not discernible in raising the level from 150 to 200° C. It showed that at 200° C. a nearly complete conversion was achieved after ca. 5 hours, while at 150° C. very little product develops.

### Use of Vacuum or Stripping Gas (Nitrogen)

Driving off the ammonia formed from the reaction mixture by vacuum or stripping with nitrogen was identified as the main parameter influencing the reaction of urea with polyethyleneglycol. Operation under vacuum was investigated with two tests at a pressure of 300 mbar. A marked improvement of the conversion behavior could be established in comparison to the reaction without driving off the ammonia formed at ambient pressure. Still better results were obtained in the gas treatment of the reaction mixture with nitrogen. Variation of the stream volume had a clear influence on the reaction of urea with polythyleneglycol.

Through the method in accordance with the invention the production of a mixture of high molecular weight organic carbonates and carbamates is possible, which can be employed as auxiliary materials or intermediate products for a range of chemical syntheses, e.g. for the manufacture of organic carbonates.

An important influencing value for the obtaining of higher conversions is the volume of the stripping gas stream. For sufficiently high volume streams the removal of the ammonia is no longer the rate-determining step.

The conversion of the mixture of carbonates or carbamates of the polymeric alcohols produced in the first stage with methanol proceeds relatively quickly with a basic catalyst, with use of a slightly increased pressure of ca. 6 bar at a temperature of ca. 140° C. Equilibrium is established after less than 1 hour in batch operation. As catalyst a quaternary ammonium salt was employed, which showed good catalytic properties. Still higher reaction rates were achieved through use of magnesium methylate.

In a coupling of both method stages after separation of the dimethyl carbonate or the diphenyl carbonate, the polymeric alcohol used as auxiliary alcohol is fed back again into the first method stage. By means of operating the method in the loop the losses of the polymeric auxiliary alcohol are prevented, so that the method is to be considered as unusually economical.

1. An intermediate product comprised of a mixture of organic carbonates and carbamates, characterized in that they are manufactured through reaction of urea, a substituted urea, a salt or ester of carbamic acid or one of their N-substituted derivatives with polymeric multifunctional alcohols, like polyalkyleneglycols, polyester polyols, or polyether polyols of general formula I:
H—[—O—R—]ₙ—OH (formula I)
in which R stands for a straight chain or branched chain alkylene group having 2 to 12 carbon atoms and n is a number between 2 and 20,
or having complete or partially hydrolyzed polyvinylalcohols of general formula II 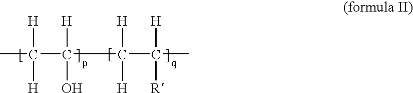
in which R′ stands for an alkyl, aryl or acyl group having 1-12 carbon atoms, p and q are numbers between 1 and 20,
or with mixtures of these compounds, without or in the presence of a catalyst favoring splitting off of ammonia.

2. A method for the manufacture of organic carbonates and carbamates, characterized in that urea, a substituted urea, a salt or ester of carbamic acid or one of their N-substituted derivatives
in a first stage with polymeric multifunctional alcohols like polyalkyleneglycols, or polyether polyols of general formula I
H—[—O—R—]ₙ—OH (formula I)
in which R stands for a straight chain or branched alkylene group having 2 to 12 carbon atoms and n is a number between 2 and 20, or
or completely or partially hydrolyzed polyvinylalcohols of general formula II 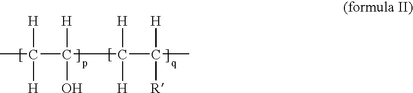
in which R′ stands for an alkyl, aryl or acyl group having 1-12 carbon atoms, p and q are numbers between 1 and 20,
or dissolved in mixtures of these compounds, without or in the presence of an ammonia splitting favorable catalyst is converted to a carbonate and carbamate containing mixture,
and at the same time the thereby liberated ammonia or the amine is removed from the reaction mixture by means of a stripping gas and or steam and/or vacuum.

3. The method according to claim 2, characterized in that the conversion to the intermediate product in accordance with the invention is carried out at temperatures between 100° C. and 270° C.

4. The method according to claims 2 and 3, characterized in that the alkaline reacting salts, oxides, hydroxides, alcoholates with elements of groups Ia, Ib, IIa, IIb, IIIa, IIIb, IVa, IVb, Va, Vb, VIb, VIIb, VIIIb of the Periodic System, basic zeolites, polymeric ion exchangers or tetraalkylammonium salts or triphenylphosphines or tertiary amines are employed as catalysts.
